(19) United States
(12) Patent Application Publication
(10) Pub. No.: US 2013/0296277 A1
(43) Pub. Date: Nov. 7, 2013

(54) Pharmaceutical Compositions for the Treatment of Parasitic Diseases, Cancer, or Skin Diseases by Topical Administration

(71) Applicants: Frans Herwig Jansen, Oud-Turnhout (BE); Annie Marie Fortin, Montreal (CA)

(72) Inventors: Frans Herwig Jansen, Oud-Turnhout (BE); Annie Marie Fortin, Montreal (CA)

(73) Assignee: DAFRA PHARMA RESEARCH & DEVELOPMENT BVBA, Turnhout (BE)

(21) Appl. No.: 13/979,503

(22) PCT Filed: Jan. 12, 2012
(86) PCT No.: PCT/EP12/50456

§ 371 Date: Jul. 12, 2013

(30) Foreign Application Priority Data
Jan. 14, 2011 (EP) PCT/EP2011/050442

(51) Int. Cl. A61K 31/661; A61K 9/00

(52) U.S. Cl. CPC A61K 31/661; A61K 9/0014
USPC 514/114

(57) ABSTRACT

The present invention relates to pharmaceutical compositions for the treatment of parasitic diseases, cancer, or skin diseases by topical administration and especially ointments for the treatment of leishmaniasis. The present invention further relates to the use of the present ointments for the treatment of parasitic diseases, cancer, or skin diseases and especially ointments for the treatment of leishmaniasis in both humans and animals. Specifically, the present invention relates to pharmaceutical compositions such as ointments and crèmes for the treatment of parasitic diseases, cancer, or skin diseases by topical administration comprising 0.1 to 20 weight %, preferably 0.1 to 10 weight %, more preferably 0.1 to 7 weight %, most preferably 0.5 to 5 weight %, of the pharmaceutical composition oleyl phosphocholine as an active ingredient.

The present invention relates to pharmaceutical compositions such as ointments and crèmes for the treatment of parasitic diseases, cancer, or skin diseases by topical administration and especially pharmaceutical compositions for the treatment of leishmaniasis. The present invention further relates to the use of the present pharmaceutical compositions for the treatment of parasitic diseases, cancer, or skin diseases and especially pharmaceutical compositions for the treatment of leishmaniasis in both humans and animals.

Miltefosine (MIL) belongs to the chemical group of alkylphosphocholines and is generally used for the treatment of visceral leishmaniasis (VL). Its particular advantages are an oral route of administration and no cross-resistance is observed with any other first- and second-line anti-leishmaniasis therapy.

Although three drugs or drug formulations of miltefosine (liposomal amphotericin B, miltefosine and paromomycin) are currently available for the treatment of leishmaniasis, they all suffer from limitations of cost, toxicity and/or the need for parenteral administration.

An alternative for miltefosine is the alkylphosphocholine oleyl phosphocholine (C18:1-PC), or OlPC providing, amongst others, a more effective treatment of parasitic diseases such as leishmaniasis and malaria both in humans and animals.

Although the use of oleyl phosphocholine for the treatment of several diseases is suggested, the suggested formulations of oleyl phosphocholine are solutions, suspensions or emulsions. Especially for topical administration, there is a need in the art to provide oleyl phosphocholine in the form of pharmaceutical composition which can be used for topical administration.

It is an object of the present invention, amongst other objects, to provide dosage forms of oleyl phosphocholine for topical administration and especially dosage forms of oleyl phosphocholine in the form of an ointment or crème suitable for both humans and animals.

The above objects, amongst other objects, are met by an ointment of oleyl phosphocholine for topical administration as defined in the appended claim 1.

Specifically, the above objects, amongst other objects are, according to a first aspect of the present invention, met by pharmaceutical compositions for topical administration comprising 0.1 to 20 weight %, preferably 0.1 to 10 weight %, more preferably 0.1 to 7 weight %, most preferably 0.5 to 5 weight % such as 0.5, 1, 1.5, 2, 2.5, 3, 3.5, 4 or 4.5 weight %, of the composition oleyl phosphocholine as an active ingredient and pharmaceutically acceptable carriers and excipients for topical administration.

The present inventors have surprisingly discovered that oleyl phosphocholine, administered topically, has significant beneficial effects when compared to placebo and miltefosine at equivalent dosages.

According to a preferred embodiment, the present pharmaceutical compositions are in the form of an ointment, crème or spray.

According to another preferred embodiment, the present pharmaceutical compositions comprise as pharmaceutically acceptable carriers and excipients:
1 to 20 weight % of the pharmaceutical composition of one or more stabilizers;
0.5 to 5 weight % of the pharmaceutical composition of one or more non-ionic surfactants;
7 to 45 weight % of the pharmaceutical composition of a semi-solid mixture of hydrocarbons;
0.05 to 0.6 weight % of the pharmaceutical composition of one or more preservatives; and
water up to 100 weight % of the pharmaceutical composition.

According to yet another preferred embodiment, the present pharmaceutical compositions comprise:
1 to 20 weight % of the pharmaceutical composition of one or more stabilizers;
0.5 to 5 weight % of the pharmaceutical composition of one or more non-ionic surfactants;
5 to 30 weight % of the pharmaceutical composition Vaseline;
2 to 15 weight % of the pharmaceutical composition paraffin;
0.05 to 0.6 weight % of the pharmaceutical composition of one or more preservatives; and
water up to 100 weight % of the pharmaceutical composition.

The more stabilizers according to the present invention are preferably selected from the group consisting of cetyl alcohol, stearyl alcohol and cetostearyl alcohol.

The one or more non-ionic surfactants according to the present invention are at least cetomacrogol 1000.

The one or more preservatives according to the present invention are at least potassium sorbate.

According to an especially preferred embodiment, the present pharmaceutical compositions comprise:
0.1 to 7 weight % of the pharmaceutical composition oleyl phosphocholine;
4 to 10 weight % of the pharmaceutical composition of one or more emulsion stabilizers;
1 to 3 weight % of the pharmaceutical composition of one or more non-ionic surfactants;
10 to 20 weight % of the pharmaceutical composition vaseline;
4 to 10 weight % of the pharmaceutical composition paraffin;
0.2 to 0.4 weight % of the pharmaceutical composition of one or more preservatives; and
water up to 100 weight % of the pharmaceutical composition.

According to yet another especially preferred embodiment, the present pharmaceutical compositions comprise:
0.5 to 7, preferably less than 5, weight % of the pharmaceutical composition oleyl phosphocholine;
6 to 8, preferably 7, weight % of the pharmaceutical composition of one or more emulsion stabilizers;
1 to 3, preferably 2, weight % of the pharmaceutical composition of one or more non-ionic surfactants;
14 to 16, preferably 15, weight % of the pharmaceutical composition Vaseline;
5 to 7, preferably 6, weight % of the pharmaceutical composition paraffin;
0.2 to 0.4, preferably 0.3, weight % of the pharmaceutical composition of one or more preservatives; and
water up to 100 weight % of the pharmaceutical composition.

According to the present invention, the present Vaseline is preferably white Vaseline.

According to the present invention, the present paraffin is liquid paraffin.

According to a second aspect, the present invention relates to the use of the present pharmaceutical compositions for the treatment of parasitic diseases, cancer, or skin diseases.

According to a third aspect, the present invention relates to oleyl phosphocholine or the present pharmaceutical compositions for the treatment of parasitic diseases, cancer, or skin diseases, preferably leishmaniasis, by topical administration.

The present invention will be further detailed in the following example of preferred embodiments of the present invention. In the example, reference is made to figures wherein:

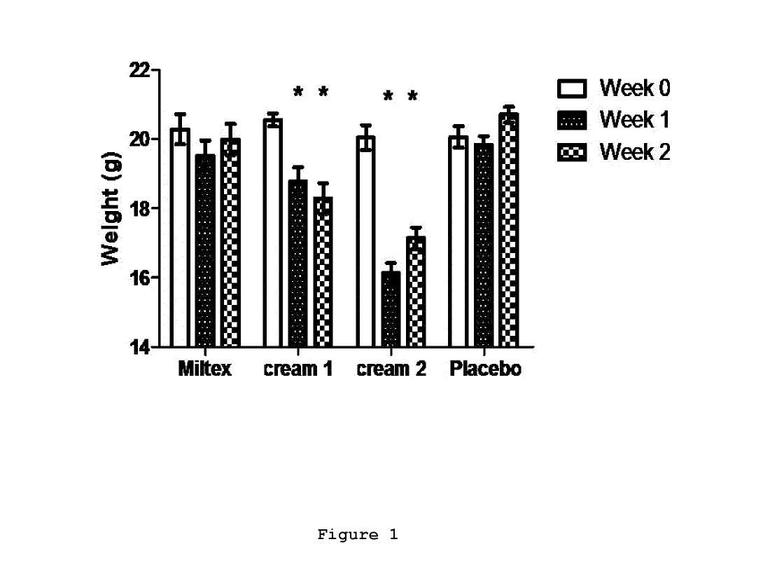

FIG. 1: shows Effect of the treatment on the total body weight. BALB/c (10-11 females/group) were infected with (5×10⁶ parasite/50 μl) Leishmania major stably transfected with luciferase. After four weeks of infection (apparent lesion) mice were treated daily for ten days (except weekends) with different compounds (Miltex, Cream 1, Cream 2 and Placebo). Following, body weight was monitored before (week 0), after week 1 and at week 2 of treatment. *p<0.05 compared week 1 or week 2 versus before treatment (week 0).

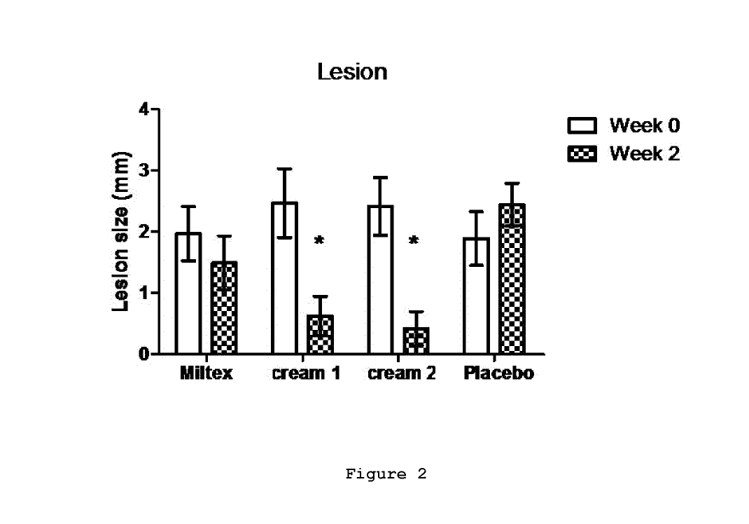

FIG. 2: shows the effect of the treatment on the size of the lesion. BALB/c (10-11 females/group) were infected with (5×10⁶ parasite/50 μl) Leishmania major stably transfected with luciferase. After four weeks of infection (apparent lesion) mice were treated daily for ten days (except weekends) with different compounds (Miltex, Cream 1, Cream 2 and Placebo). Following, size of the lesion was measured before (week 0) and at week 2 of treatment. *p<0.05 compared week 2 versus before treatment (week 0).

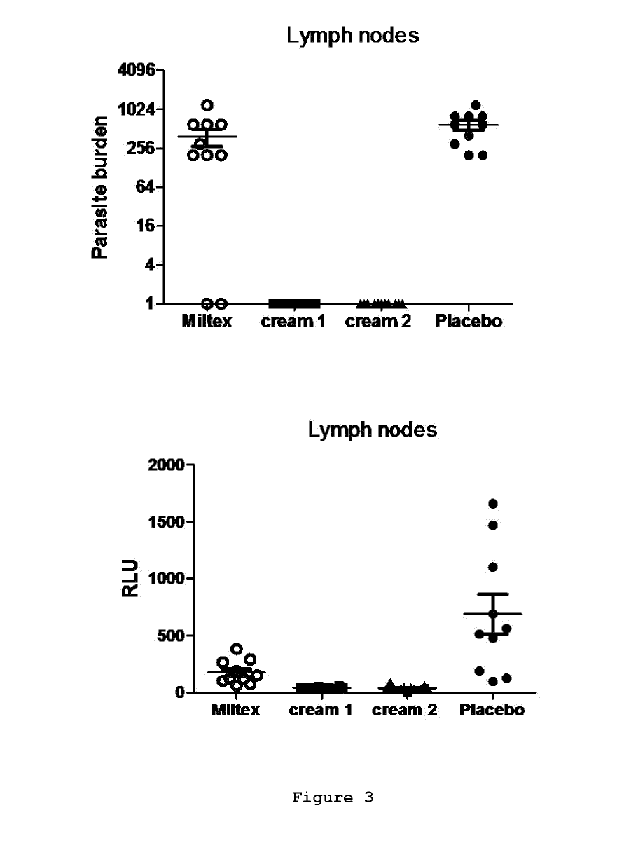

FIG. 3: shows parasite burden in the draining lymph nodes. BALB/c (10-11 females/group) were infected with (5×10⁶ parasite/50 μl) Leishmania major stably transfected with luciferase. After four weeks of infection (apparent lesion) mice were treated daily for ten days (except weekends) with different compounds (Miltex, Cream 1, Cream 2 and Placebo). Following 2 weeks of treatment mice were sacrificed and parasite burden in the lymph nodes was monitored using limiting dilution (A) and by luciferase activity (B).

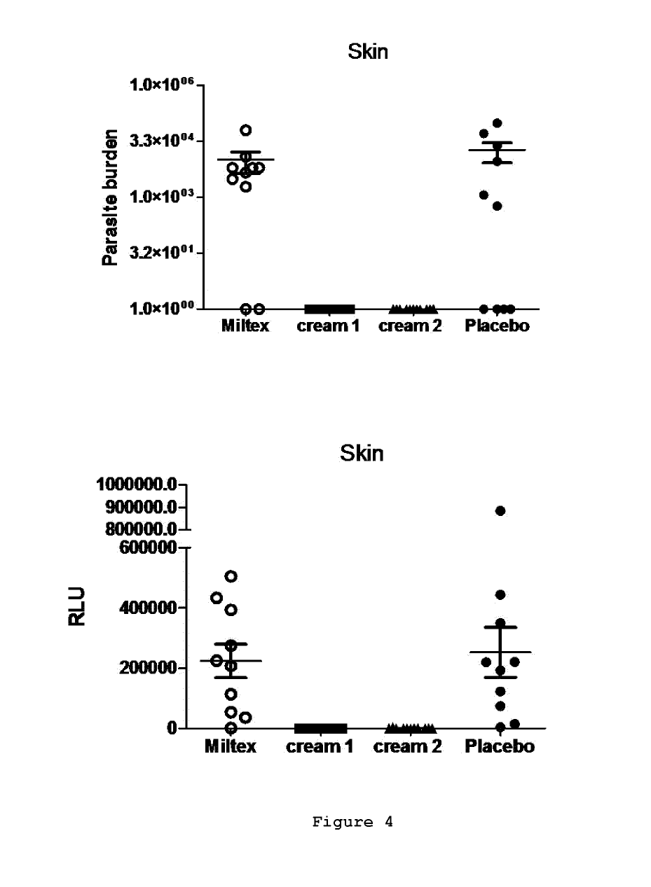

FIG. 4: shows parasite burden at the infection site (skin). BALB/c (10-11 females/group) were infected with (5×10⁶ parasite/50 μl) Leishmania major stably transfected with luciferase. After four weeks of infection (apparent lesion) mice were treated daily for ten days (except weekends) with different compounds (Miltex, Cream 1, Cream 2 and Placebo). Following 2 weeks of treatment, mice were sacrificed and parasite burden was monitored at the site of infection (skin) using limiting dilution (A) and by luciferase activity (B).

### EXAMPLE

### Comparison Between Topical Administration of OlPC and Miltefosine (Miltex®) in Balb/c Mice Infected with L. Major

### Objectives

To validate the efficacy of Miltex® to treat L. major-derived cutaneous lesions in Balb/c infected female mice. To compare the efficacy of Miltex® to the one of OlPC on an experimental model of cutaneous leishmaniasis. To evaluate possible local or systemic toxicity due to the treatment with Miltex® and OlPC compared to placebo.

### Experimental Design

At day 0, 10 Balb/c female mice per group were infected subcutaneously (s.c.) using 5×10⁶ stationary-phase L. major-LUC promastigotes at the tail base and development of lesion was followed on a weekly basis. Four weeks later, treatment of the established lesions was initiated with active anti-leishmania compounds or with empty vehicle.

Twenty-five μl of Miltex (1.5 mg HePC) and about 27 mg of the OlPC ointment (1.5 mg OlPC) was applied daily directly on to the lesions for 5 days a week, during 2 weeks. The parasite burden of draining lymph nodes (inguinal and lower periaortic) and skin lesions was assessed immediately after the end of treatment. Mice were treated (see administration routes in Table 1) starting from day +31 to day +42 post infection, 5 days/week. Drug application was performed around 10 h. am.

At the end of the treatment period, parasite burden was evaluated by dilution assay and luciferase activity. In addition, lesion size was measured on a weekly basis using an electronic caliper. Animal weights were also followed.

TABLE 1
Mice to be used for treatment

| Initial | |
|---|---|
| Dose | |
| (mg/day) | Number of Mice |

| Group Designation | API | Males | Females |
|---|---|---|---|
| 1. Miltex ® | 1.5 | N/A | Balb/c |
| | | | x10 |
| 2. OlPC 6% | 1.5 | N/A | Balb/c |
| | | | x10 |
| 3. OlPC 6% + 0.1% | 1.5 | N/A | Balb/c |
| betamethasone valerate | | | x10 |
| 4. Placebo | N/A | N/A | Balb/c |
| | | | x10 |

Mice were treated with the indicated dose on a daily basis.

### Test and Control/Vehicle Articles

| | |
|---|---|
| Identity: | Oleylphosphocholine (OlPC) ointment |
| | formulation |
| Chemical Name: | cis-9-Octadecenylphosphocholine |
| | (C₂₃H₄₈NO₄P) |
| Description: | White, creamy with oily film |
| Batch/Lot No.: | 01100504 |
| Retest Date: | N/A |
| Purity: | 6% |
| Storage | 2-8° C. in original package |
| Conditions: | |

| | |
|---|---|
| Identity: | 6% Oleylphosphocholine (OlPC) + 0.1% |
| | betamethasone valerate ointment |
| | formulation |
| Chemical Names: | cis-9-Octadecenylphosphocholine |
| | (C₂₃H₄₈NO₄P) |
| | betamethasone (C₂₂H₂₉FO₅) |
| Description: | White, creamy with oily film |
| Batch/Lot No.: | 01102206 |
| Retest Date: | N/A |
| Purity: | 6% + 0.1% |
| Storage | 2-8° C. in original package |
| Conditions: | |

| | |
|---|---|
| Identity: | Miltex ® |
| Chemical Name: | Hexadecylphosphocholine |
| | (C₂₁H₄₆NO₄P) |
| Description: | Oily liquid, clear and colorless |
| Batch/Lot No.: | 91087 |
| Retest Date: | September 2012 (expiry date) |
| Purity: | 60 mg/mL |
| Storage | Room temperature, in original package |
| Conditions: | |
| Supplier: | Baxter Oncology GmbH |

| | |
|---|---|
| Identity: | cetromacrogol base (placebo) |
| Chemical Name: | N/A |
| Description: | White, creamy, relatively thick |
| Batch/Lot No.: | N/A |
| Retest Date: | N/A |
| Purity: | N/A |
| Storage | 2-8° C. in original package |
| Conditions: | |

### Pre-Treatment

40 BALE/c female mice (6-8 weeks) were ordered from Charles River. Mice were separated by cages, maximum 5 animals per cage, and provided with sterile water and food. Then mice were given some time to acclimatize to the Animal Facility conditions.

Mice were injected with 5×10⁶ parasites/50 μl of Leishmania major stably transfected with luciferase using a 1 ml syringe with a 27 G^{1/2} needle. Mice were then checked regularly to monitor the appearance of the lesion. Subsequently, after a visual inspection, it was decided that the lesions were adequate based on their size, appearance and development and treatment was initiated.

Mice were individually weighted. Hair at the lesion site was removed using a shaver. The size of their lesion was individually monitored using a mechanical caliper. Cages were assigned to a certain group based on the fact that we aimed for a similar group weight average (around 20 grams), as well as a similar lesion development success (Miltex®/Group 1: 7 mice out of 10, Cream 1/Group 2: 7 mice out of 10, Cream 2/Group 3: 8 mice out of 11, Placebo/Group 4: 7 mice out of 10).

Mice were treated daily from day+31 to day+35. No treatment was applied during the weekend. The daily treatment re-started on day+38 day+42. Mice were treated for a total of 10 days.

Miltex® (Hexadecylphosphocholine) (25 μl) was measured using a p200 Gilson pipetman and sterile plastic tips. As for Cream 1 (cis-9-Octadecenylphosphocholine), Cream 2 (cis-9-Octadecenylphosphocholine betamethasone) and Placebo (cetromacrogol base), “the size” of 27mg was determined using a precision scale, and this was used as the standard amount to treat the lesion of the mice (as per study protocol, section 3.3.). A surface corresponding to 1.5 cm×1.5 cm, on and around the lesion, was treated with the different products. A light massage was performed in order to facilitate the penetration of the products.

Everyday before the treatment, mice were quickly observed for any signs of discomfort or distress (for example: redness, weight loss, excess scratching). The weight and the size of the lesion were also monitored after one week of treatment as well as at the end of the study. Before every size lesion measurement hair at the site of the lesion were removed. Pictures were also taken.

Mice were sacrificed using CO₂ chamber and they were sprayed with ethanol on their anterior side. Cardiac puncture was immediately performed to collect the blood using a sterile syringe and a 27 G^{1/2} needle. The tubes containing the blood were put on ice, to be later processed. Draining lymph nodes were removed, put in a tube containing cold sterile PBS and then put on ice. The peritonea of the mice were open in order to visually inspect the internal organs.

Clidox was used to disinfect the lesion region. After 5 minutes, that region was sprayed with ethanol, to remove excess of Clidox. The treated zone (square of skin) was then removed and put in a tube containing cold sterile PBS.

Blood was left on ice to coagulate and then the tubes were spun at 13 000 rpm for 30 minutes. Serum was transferred and aliquoted in new-labeled tubes. The tubes were put at −80° C.

Draining lymph nodes were washed four times with cold sterile PBS and they were crushed between two sterile microscope slides. Cells were recuperated using cold sterile PBS. 200 μl was used to perform the dilution assay, 100 μl per 1^{st} well of a 96-well plate (2-fold dilution) as the experiment was done in duplicate. This 100 μl was mixed with 100 μl of SDM (parasite media), resuspended 5 times and 100 μl of this mix was transferred to the next well, and so on. Plates were sealed with parafilm and incubated at 25° C. for 7 days.

The remaining cells in the tube were spun down at 2 500 rpm for 10 minutes, and the supernatant was removed. Pellets were kept at −80° C. until the luciferase assay was performed. Briefly, pellets from lymph nodes were thawed on ice and resuspended in 50 ul of lysis buffer. Tubes were vortexed, left on ice for 20 minutes and then vortexed again. Finally the tubes were centrifuged at 3000 rpm for 15 minutes and supernatant was collected. 20 μl of this supernatant and 90 μl of luciferase assay reagent were mixed to obtain the luciferase counts using a Mini Lumat LB 9506 luminometer. Readings were done in duplicate. Results are expressed in Relative Light Units (RLU).

Pieces of skin were washed four times with cold sterile PBS and they were disrupted using a Dounce homogenizer. Cells were recuperated using cold sterile PBS. 200 μl were used to perform the dilution assay, 100 μl per 1^{st} well of a 96-well plate (2-fold dilution) as the experiment was done in duplicate, as described in the previous paragraph. The remaining cells were spun down at 2 500 rpm for 10 minutes, and the supernatant was removed. Pellets were kept at −80° C. until the luciferase assay was performed.

All individual data were compiled in Excel worksheet. Average and SEM for weight loss, lesion size variation, parasite burden and luciferase activity were calculated from the raw data. Graphs were done using Prism.

### Results

No pain or distress was noticed during the first week of treatment, as the animals responded very well to the treatment. No distress was observed also during the second week, but pain/sensibility was. The treated zone seemed to be sensitive when the treatment was applied, especially in the case of Miltex®. Excessive scratching wasn't observed when the mice were treated, or right after the treatment.

For the first week of treatment, the mice responded very well. The lesion of most individuals almost completely disappeared after only two or three treatments.

Later, the mice treated with Miltex®, Cream 1 and Cream 2 developed really strong skin reactions (irritation/corrosion) over the weekend. The skin reactions were scored using the OECD grading of skin reactions. These observations are of course subjective. These results are shown in Table 2.

TABLE 2
Score of the skin damages observed during the treatment period.

| Group | Mice | | Value | |
|---|---|---|---|---|
| # | # | Treatment | Week 1 | Week 2 |
| 1 | 1.1 | Miltefosine (MILTEX ®) | 4 | 4 |
| | 1.2 | | 4 | 4 |
| | 1.3 | | 4 | 4 |
| | 1.4 | | 4 | 4 |
| | 1.5 | | 4 | 4 |
| 2 | 2.1 | Oleylphosphocholine (OlPC) | 4 | 4 |
| | 2.2 | ointment formulation - | 4 | 4 |
| | 2.3 | Cream 1 | 4 | 4 |
| | 2.4 | | 4 | 4 |
| | 2.5 | | 4 | 4 |
| 3 | 3.1 | Oleylphosphocholine (OlPC) | 4 | 4 |
| | 3.2 | ointment formulation - | 4 | 4 |
| | 3.3 | Cream 1 | 4 | 4 |
| | 3.4 | | 4 | 4 |
| | 3.5 | | 4 | 4 |
| 4 | 4.1 | 6% Oleylphosphocholine | 3 | 4 |
| | 4.2 | (OlPC) + 0.1% | 3 | 4 |
| | 4.3 | betamethasone valerate | 3 | 4 |
| | 4.4 | ointment formulation - | 3 | 4 |
| | 4.5 | Cream 2 | 3 | 4 |
| 5 | 5.1 | Miltefosine (MILTEX ®) | 4 | 4 |
| | 5.2 | | 4 | 4 |
| | 5.3 | | 4 | 4 |
| | 5.4 | | 4 | 4 |
| | 5.5 | | 4 | 4 |
| 6 | 6.1 | Cetromacrogol base - | 0 | 0 |
| | 6.2 | Placebo | 0 | 0 |
| | 6.3 | | 0 | 0 |
| | 6.4 | | 0 | 0 |
| | 6.5 | | 0 | 0 |
| 7 | 7.1 | Cetromacrogol base - | 0 | 0 |
| | 7.2 | Placebo | 0 | 0 |
| | 7.3 | | 0 | 0 |
| | 7.4 | | 0 | 0 |
| | 7.5 | | 0 | 0 |
| 8 | 8.1 | 6% Oleylphosphocholine | 3 | 4 |
| | 8.2 | (OlPC) + 0.1% | 3 | 4 |
| | 8.3 | betamethasone valerate | 3 | 4 |
| | | ointment formulation - | | |
| | | Cream 2 | | |
| 9 | 9.1 | 6% Oleylphosphocholine | 3 | 4 |
| | 9.2 | (OlPC) + 0.1% | 3 | 4 |
| | 9.3 | betamethasone valerate | 3 | 4 |
| | | ointment formulation - | | |
| | | Cream 2 | | |

BALB/c (10-11 females/group) were infected with (5 × 10⁶ parasite/50 μl) Leishmania major stably transfected with luciferase. After four weeks of infection (apparent lesion) mice were treated daily for ten days (except weekends) with different compounds (Miltex ®, Cream 1, Cream 2 and Placebo). Following, skin damages were monitored after week 1 and at week 2 of treatment. This grading was done in accordance to the OECD grading of skin reactions; OECD guideline for testing of chemicals #404, adopted Apr. 24^{th}, 2002. 0 = No erythema; 1 = Very slight erythema; 2 = Well-defined erythema; 3 = Moderate to severe erythema; 4 = Severe erythema (beef redness) to slight eschar formation (injuries in depth).

It was also noticed that mice receiving Cream 2 looked like they lost a lot of weight. All mice were weighted to make sure that they didn't lost more than 20% of their initial total body weight (ITBW). After calculation it was found that Miltex®/Group 1 mice lost 3.75% of their ITBW, Cream 1/Group 2 mice lost 8.64% of their ITBW, Cream 2/Group 3 mice lost 19.50% of their ITBW and Placebo/Group 4 mice lost 1.14% of their ITBW. Raw data used to do these calculations are shown in Annex 1 (week 0) and Annex 2 (week 1). Since the mice from Group 2 were really close to the limit permitted, but didn't reach it, the decision was taken to pursue the study.

By the end of the study, the ITBW of each group was calculated again. Overall mice from Miltex®/Group 1 lost 1.46% of their ITBW, mice from Cream 1/Group 2 lost 11.02% of their ITBW, mice from Cream 2/Group 3 lost 14.47% of their ITBW and mice from Placebo/Group 4 gained 3.2% of their ITBW. FIG. 1 shows the effect of the different treatments on the total body weight of the mice.

After two weeks of treatment, reduction in the average size of the lesion was quite significant for groups treated with Cream 1 (Group 2) or Cream 2 (Group 3). The average lesion size from Group 2 went from 2.464 to 0.622. As for Group 3, the initial average of 2.41 was reduced to 0.415. Mice treated with Miltex® (Group 1) also saw a reduction in the average size of their lesion, but in a non-significant manner, passing from 1.966 to 1.49 after two weeks of treatment. As expected, mice treated with the Placebo Cream 3 (Group 4) saw their average lesion size increase from 1.887 to 2.445. FIG. 2 depicts the average size modification of the Leishmania-induced lesion by the different treatments.

Organs (kidney, liver and spleen) were inspected after euthanasia. Pictures were also taken. No major group-related significant difference was noticed (size, color or aspect) for these internal organs.

Parasite burden at the site of infection (skin) and in the draining lymph nodes was measured using two different techniques; dilution assay and luciferase assay.

Parasite burden in the draining lymph nodes from mice treated with Cream 1 and Cream 2 was not detected, as we were unable to find any parasite alive, even in the first dilutions (FIG. 3; top panel). Luciferase results from these two groups were also similar, as an average of 41.7 (Cream 1) or 39.45 (Cream 2) is very close to the background reading and can be considered negligible (FIG. 3; bottom panel).

Only 390 parasites were found in the lymph nodes of mice treated with Miltex®, as this average was of 590 parasites in their placebo treated counterpart (FIG. 3; top panel). For luciferase results, the average was of 176.7 RLU for mice treated using Miltex® and 688.9 RLU for mice treated with the placebo (FIG. 3; bottom panel).

Parasite burden at the site of infection (skin) was much higher for mice treated with Miltex®, and of course for mice that received the Placebo treatment, compared to the results obtained in the draining lymph nodes. An average of 10810 parasites were found for the Miltex® treated mice, as that average was of 19380 parasites for the mice that were treated with the Placebo cream. No parasite was detected in the plates of Group 2 (Cream 1) and Group 3 (Cream 2). FIG. 4, top panel, shows the results of the parasite burden at the site of infection (skin) using the dilution assay.

Luciferase activity was detected for all groups. The RLU average was of 224690.4 RLU for mice treated with Miltex®, 352.6 RLU for the one treated with Cream 1, 591.2 RLU for Cream 2 treated mice and finally, 253221.2 RLU for mice treated with the Placebo cream. FIG. 4, bottom panel, shows the results of the luciferase activity at the site of infection (skin) for each group.

### CONCLUSION

The objectives of this example were:
To validate the efficacy of Miltex® to treat L. major-derived cutaneous lesions in BALB/c infected female mice.
To compare the efficacy of Miltex® to the one of OlPC on an experimental model of cutaneous Leishmaniasis.
To evaluate possible local or systemic toxicity due to the treatment with Miltex® or OlPC compared to placebo.

Using Miltex®, a reduction was observed in the size of the lesion, as well as in the parasite burden, compare to the results of the Placebo-treated mice. However, this reduction was not significant. Mice that were treated with Miltex® also showed a really strong skin reaction after week 1, that irritation/corrosion lasted until the end of the study.

Mice that were treated with the Oleylphosphocholine (OlPC) ointment formulation or the 6% Oleylphosphocholine (OlPC)+0.1% betamethasone valerate ointment formulation showed a significant reduction in the size of their lesion, as well as no or very low parasite burden. This shows that these products are more efficient than Miltex®.

1. Pharmaceutical composition for topical administration comprising 0.1 to 20 weight %, of the pharmaceutical composition oleyl phosphocholine as an active ingredient and pharmaceutically acceptable carriers and excipients for topical administration.

2. Pharmaceutical composition according to claim 1 in the form of an ointment, crème or spray.

3. Pharmaceutical composition according to claim 1 comprising as pharmaceutically acceptable carriers and excipients:
1 to 20 weight % of the pharmaceutical composition of one or more stabilizers;
0.5 to 5 weight % of the pharmaceutical composition of one or more non-ionic surfactants;
7 to 45 weight % of the ointment of a semi-solid mixture of hydrocarbons;
0.05 to 0.6 weight % of the pharmaceutical composition of one or more preservatives; and
water up to 100 weight % of the pharmaceutical composition.

4. Pharmaceutical composition according to claim 3 comprising:
1 to 20 weight % of the pharmaceutical composition of one or more stabilizers;
0.5 to 5 weight % of the pharmaceutical composition of one or more non-ionic surfactants;
5 to 30 weight % of the pharmaceutical composition Vaseline;
2 to 15 weight % of the pharmaceutical composition paraffin;
0.05 to 0.6 weight % of the pharmaceutical composition one or more preservatives; and
water up to 100 weight % of the pharmaceutical composition.

5. Pharmaceutical composition according to claim 3 wherein the one or more stabilizers are selected from the group consisting of cetyl alcohol, stearyl alcohol and cetostearyl alcohol.

6. Pharmaceutical composition according to claim 3, wherein the one or more non-ionic surfactants are at least cetomacrogol 1000.

7. Pharmaceutical composition according to claim 3, wherein the one or more preservatives are at least potassium sorbate.

8. Pharmaceutical composition according to claim 4, comprising:
0.1 to 7 weight % of the pharmaceutical composition oleyl phosphocholine;
4 to 10 weight % of the pharmaceutical composition of one or more emulsion stabilizers;
1 to 3 weight % of the pharmaceutical composition of one or more non-ionic surfactants;
10 to 20 weight % of the pharmaceutical composition vaseline;
4 to 10 weight % of the pharmaceutical composition paraffin;
0.2 to 0.4 weight % of the pharmaceutical composition of one or more preservatives; and
water up to 100 weight % of the pharmaceutical composition.

9. Pharmaceutical composition according to claim 8, comprising:
0.5 to 7 weight % of the pharmaceutical composition oleyl phosphocholine;
6 to 8 weight % of the pharmaceutical composition of one or more emulsion stabilizers;
1 to 3 weight % of the pharmaceutical composition of one or more non-ionic surfactants;
14 to 16 weight % of the ointment Vaseline;
5 to 7 weight % of the pharmaceutical composition paraffin;
0.2 to 0.4 weight % of the pharmaceutical composition of one or more preservatives; and
water up to 100 weight % of the pharmaceutical composition.

10. Pharmaceutical composition according to claim 4, wherein said Vaseline is white Vaseline.

11. Pharmaceutical composition according to claim 4, wherein said paraffin is liquid paraffin.

12. (canceled)

13. (canceled)

14. A method of treating parasitic diseases, cancer, or skin disease of a human or animal, comprising administering the pharmaceutical composition according to claim 1 to a human or animal in need of treatment.

15. The method of claim 14, wherein the disease is leishmaniasis.

16. The method of claim 14, wherein the pharmaceutical composition is topically administered.
